(19) United States
(12) Patent Application Publication
(10) Pub. No.: US 2005/0042661 A1
(43) Pub. Date: Feb. 24, 2005

(54) Use of novel compounds to release nucleotides from living cells

(76) Inventors: Veikko Tarkkanen, Monchen Gladbach (DE); Andreas Schafer, Kerken (DE); Andrew Hearn, Chicago, IL (US)

Correspondence Address: NovusIP, LLC, Suite 221, 521 West Superior, Chicago, IL, 60610, US

(21) Appl. No.: 10/916,882

(22) Filed: Aug. 12, 2004

Related U.S. Application Data
(60) Provisional application No. 60/495,440, filed on Aug. 15, 2003.

(51) Int. Cl. C12Q 1/68; C12N 1/08

(52) U.S. Cl. 435/6; 435/270

(57) ABSTRACT

A method for effecting a rapid, comprehensive release of cellular contents, including nucleotides and associated molecules of interest from living cells, using a novel family of quaternary ammonium compounds. Modification of the alkyl chain length of these compounds can enable selective release from different classes of living cells, allowing them to be rapidly distinguished from each other.

### RELATED APPLICATION

This application is based on U.S. Provisional Application No. 60/495,440 titled “Use of novel compounds to release nucleotides from living cells” filed on 15 Aug. 2003.

### FIELD OF THE INVENTION

The present invention relates to a method for effecting a rapid and comprehensive release of cellular contents, including nucleotides and other associated molecules of interest from living cells.

### BACKGROUND OF THE INVENTION

Many industries and markets have a requirement to detect the presence of contamination in their products or samples. Chief among these contaminants are microbial cells, such as bacteria, yeasts or molds. These types of cells are microscopic in size, effectively impossible to detect by visible means.

Most conventional methods to detect microbial cells rely on techniques that encourage the growth of possible contaminants until the growth can be seen by the naked eye; a process that can take many days. The delays caused by this requirement are costly and even potentially dangerous.

There is therefore a need to detect microbial contamination as rapidly as possible. One approach commonly taken to effect this is to isolate and detect specific chemical markers within contaminating microbial cells. One example is the molecule Adenosine Triphosphate (“ATP”), which is universally present in all living matter, where it serves as a source of energy for processes within cells. ATP can be quickly detected using the firefly luciferase reaction—addition of ATP to a purified preparation of the firefly enzyme luciferase and its substrate luciferin will produce an instant light emission, a phenomenon known as ATP-bioluminescence. This light is proportional to the amount of ATP present, and can be measured using a sensitive light detecting instrument such as a luminometer.

Commercial systems are available, comprising luciferase-based reagent kits and luminometer instrumentation, that enable customers to quickly test their products and samples for ATP which, if found in unusual amounts, indicates the presence of microbial contamination.

One challenge to the development of tests for microbial contamination by ATP-bioluminescence is that the ATP required for detection is ‘hidden’ or contained within the contaminating cells. Hidden ATP is not available to trigger the luciferase/luciferin light reaction, so in this form the test will not work. To enable the test to work, ATP first has to be released from contaminating cells, in a process typically referred to as extraction.

A second challenge to the development of a successful ATP-bioluminescence test is that a majority of samples contain ATP of non-microbial origin. An example of this is bovine ATP in milk, derived ultimately from the udder cells of the cow. This non-microbial ATP may conceal or smother the microbial ATP that the test is designed to detect. To counter this, it is possible to pre-treat the sample being tested with an ATP-destroying enzyme. Such enzymes are commonly used in commercial kits, and are generally referred to as apyrase.

Treatment of a sample with apyrase is successful because the apyrase is able to remove only the ATP it finds free in solution. During this pre-treatment, ATP locked up inside microbial cells is kept safely away from the action of the apyrase; however, once release/extraction of this microbial ATP has occurred, it is vulnerable to apyrase degredation, and typically a proportion of microbial ATP is indeed lost to the action of apyrase before it can be read.

Extraction is usually accomplished by chemical means—the most common types are based around a family called Quaternary Ammonium Compounds (“QAC's”). QAC's were first described in U.S. Pat. No. 4,303,752. Specific examples include quaternary ethoxylated ammonium chlorides, benzyl trimethyl ammonium chloride, cetyl trimethyl ammonium bromide and other generic cationic detergents under various trade names.

Microbial ATP extraction poses additional challenges, in that microbial cells are typically protected by robust cell walls. A successful extractant should:
Release cell components such that they are available for subsequent detection and measurement by outside systems such as ATP-bioluminescence.
a Release cell components from a variety of different types of cells, with accordingly different wall compositions (See, for example, U.S. Pat. No. 5,558,986 to Lundin, which is hereby incorporated by reference).
Effect this release in as rapid manner as possible (Id.).
Effect this release in a reproducible manner.
Enable the timely, reproducible release of a maximum of cell components.
Perform the extraction in a wide potential range of sample environments—which may include, but are not limited to, milk, cream, fruit juice, oil emulsions, detergent solutions, liquid dyestuffs and combinations thereof.
Not interfere significantly with the stability of the released components (such as ATP) prior to subsequent detection and measurement.
Not interfere significantly with subsequent detection and measurement systems (such as ATP-bioluminescence).

### SUMMARY OF THE INVENTION

The present invention relates to a method for effecting a rapid and comprehensive release of cellular contents, including nucleotides and other associated molecules of interest from living cells, using a novel family of quaternary ammonium compounds. In addition, the invention relates to a method of using dimethyl-dialkyl-ammonium halides having varying alkyl chain lengths for selective extraction of microbial cell contents. Finally, the use of these novel compounds exhibits potential to effect the inactivation of ATP-destroying enzymes (used to deplete a sample of its non-microbial ATP).

The present invention demonstrates a superior ability to release ATP from bacteria and yeasts in dairy products. The present invention also demonstrates a superior ability to release ATP from property in case of personal care products incubated in growth media, but with the possible exception of Pseudomonas in certain cases.

### BRIEF DESCRIPTION OF THE DRAWING

FIG. 1 illustrates the effect of the proportion of quaternary ethoxylated amine on the reaction kinetics and level of light emulsion as a function of time after adding ionic surface active agent in bacterial sample.

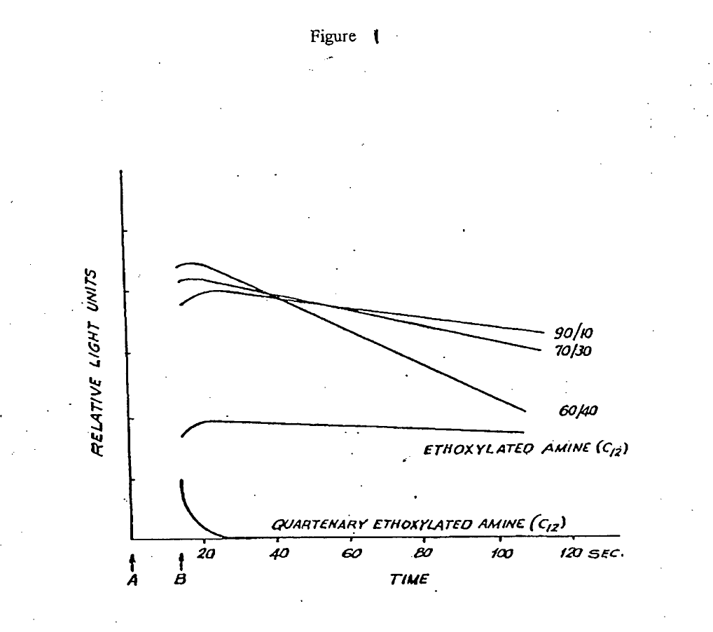

FIG. 2 illustrates the baselines for a prior art kit verses a kit in accordance with the present invention.

FIG. 3 illustrates the spiked samples for a prior art kit verses a kit in accordance with the present invention.

FIG. 4 illustrates ratios between relative light units for a prior art kit verses a kit in accordance with the present invention.

FIG. 5 illustrates the average relative light units for the different organisms tested in different products for a prior art kit verses a kit in accordance with the present invention.

FIG. 6 illustrates the signal/blank values for a prior art kit verses a kit in accordance with the present invention.

FIG. 7 illustrates the average relative light units for the different organisms tested in different products for a prior art kit verses a kit in accordance with the present invention.

FIG. 8 illustrates the signal/blank values for a prior art kit verses a kit in accordance with the present invention.

### DETAILED DESCRIPTION OF THE INVENTION

It is known that surface active agents have been used to rupture (lyze) single cells in order to eliminate ATP from somatic cells (See, for example, U.S. Pat. No. 3,745,090), but the above method has drawbacks which limit its applicability. Rupturing the cell membrane allows enzymes to be released from somatic cells and these enzymes can cause interference in the later phases of the assay of nucleotides. U.S. Pat. No. 3,745,090 suggests both non-ionic detergents (octyl phenoxy polyethoxyethanol, terpenoid saponins, steroid saponins, sulfosuccinate glycosides, and fatty acid esters of sorbitol anhydrides) and ionic detergents can be used to rupture somatic cells. Some of these, particularly the ionic surface active agents, affect the permeability of the microbial cell wall and membrane and release nucleotides (ATP, FMN and other small molecules) from microbial cells. As a result, such surfactants cannot be used for selective rupturing of somatic cells prior to measurement of microbial ATP or other nucleotides. The present invention describes methods of applying specific surface active agents for selective release of nucleotides (purine and pyridine nucleotides, and PMN) either for somatic or microbial cells. This selective release of nucleotides is accomplished without releasing enzymes from cells and the assay of nucleotides can be made without interference from undesired enzymatic hydrolysis of assayed nucleotides.

Concentration of nucleotides is measured in metabolic studies, biochemistry, clinical chemistry and bioassays. About one third of the known 2000 enzymes use purine nucleotides (ATP, ADP, AMP GTP, GMP, ITP, etc.) and pyridine nucleotides (NAD, NADH, NADP, NADPH, CTP, UTP, etc.) as substrates. In conventional methods, these substrates have been extracted from cells by destroying the cells by physical or chemical means and inactivating the enzymes in the cells by freezing, heating or with chemicals. In many conventional methods proteins have to be separated from the sample before the measurement of nucleotides. Such complicated manipulations can cause errors in the assay and make the sample preparation laborious. The present invention makes the sample preparation simple, rapid and reproducible. When these methods are used in conjunction with bioluminescent assay of metabolites, the measurement is specific to ATP, FMN, NADH OR NADPH; whichever is meant to be measured. The samples need not be deproteinized nor the nucleotides separated by chromatography or liquid extraction techniques.

Dimethyl-dialkyl-ammonium halides represent a novel class of Quaternary Ammonium compounds that can be used for efficient and effective release of cellular contents, such as nucleotides, from microbial organisms. Variation of the alkyl chain lengths of dimethyl-dialkyl-ammonium halides may affect their ability to act as nucleotide extractants. It is believed a shorter chain length of approximately 8 to 10 carbon atoms may represent a rapid, quantitative release. It is further believed that extending the chain length beyond 10 carbon atoms may allow for selectively extracting nucleotides from different microbial sources. For example, having an alkyl chain length greater than 10 carbon atoms may allow for the release of bacterial cell contents while yeast cell contents remain largely intact.

Dimethyl-dialkyl-ammonium halides also are able to inactivate apyrase enzymes while leaving luciferase enzyme molecules untouched and fully active. It is believed that this property can be used to improve and optomise current test methods—inactivating apyrase eliminates the loss of microbial ATP that currently occurs in the interval between extracting microbial contents and adding the ATP-sensitive luciferase enzyme.

### DESCRIPTION OF A PREFERRED EMBODIMENT

In the present invention, the nucleotides are released from single cells in suspension or from mono- and bilayers of cells through the cell wall and cell membrane made permeable by means of the action of surface active agents. Certain surface-active agents change the permeability of the cell membrane by affecting the integrity of the lipid and phospholipid layer in the membrane. It is possible to select certain non-ionic surface active agents which do not lyze the somatic cell but make the cell membrane permeable to small-size molecules, such as purine and pyridine nucleotides. When the cell membrane is made permeable to small molecules, nucleotides diffuse out of the cells instantaneously, but enzymes and proteins having a large molecular size will not be able to penetrate the membrane and stay inside the cells. This allows a simple and rapid extraction of nucleotides from cells without having to inactivate enzymes in the cells. Released nucleotides in extracellular solution are stable for several minutes if the solution does not have endogenous enzymes or broken cells in high enough quantities to cause a rapid, undesired enzymatic hydrolysis of nucleotides in the solution.

By selecting a non-ionic surfactant that does not lyze somatic cells but only changes the permeability of the membrane, does not affect microbial cell walls, and does not inhibit the bioluminescent reaction, it is possible to release nucleotides selectively from somatic cells for assaying by bioluminescent systems. However, microbial cells, such as bacteria, yeasts, fungi and slime molds have a cell wall that is resistant to chemical and environmental factors. The cell wall contains substances, such as muramic acid (a peptidoglycan) in bacteria, and chitinous substances in fungi which protect the more fragile cell membrane. Therefore, it is necessary to use stronger surface active agents, that is, ionic surface active agents to make the cell wall of microbes permeable for small molecules. The ionic surfactants are selected by their specific properties, such as the length of alkyl chain, degree of ethoxylation (lipophility or hydrophility) and presence of radicals, such as quaternary salts, to affect the permeability of the cell wall of microbial cells for releasing nucleotides. Since many ionic surfactants precipitate proteins and inactivate enzymes, it is necessary to use a surfactant that does not inactivate the luciferase enzyme or other enzymes used in the bioluminescent reaction, or the reaction conditions have to arranged to such that the interference from the surfactant can be eliminated, for example, by dilution.

The sample preparation for releasing nucleotides from a suspension of somatic or microbial cells requires only the mixing of the surfactant in a concentration from about 0.02 to about 2% by volume of the total combined volumes of the sample suspension and surface active agent. The diffusion of metabolites from the cells through the membrane is so rapid that even ATP, which has a turnover time in the metabolism within the cell of less than one second, can be quantitatively released.

Samples having primarily microbial cells, for example activated sludge and microbial cultures, can be extracted for nucleotides without prior elimination of nucleotides of non-microbial cells. Microbial cell walls have substances, such as peptidoglycans, chitin and mucoidal substances that make the wall resistant to chemicals. Therefore, it is more difficult to release nucleotides from microbial cells than from somatic cells. According to the present invention, the application of ionic surface active agents changes the permeability of the cell wall and membrane of microbes to make them permeable for small size molecules, such as nucleotides, but not for enzymes. By “ionic surface active agents” is meant anionic or cationic surface active agents to the exclusion of non-ionic surface active agents. This selective permeability is obtained by treating microbes with ionic surface active agents, the best of which are those that contain quaternary ammonium salts and a fatty group with a chain length of 12 carbon atoms; however, any chain length of carbon atoms from 8 to 18 can be used.

Examples of suitable ionic surfactants for quantitative release of nucleotides from microbial cells for bioluminescent measurement with firefly and photobacterial systems are: ethoxylated amines, ethoxylated diamines, polyethylene glycol esters of fatty acids, and ethoxylated amides having a chemical structure of: 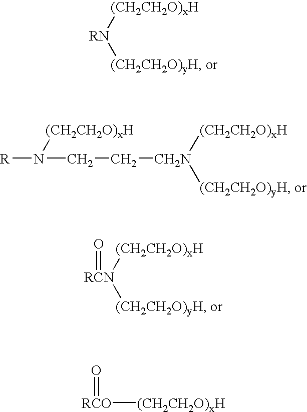
where R is a fatty alkyl group having 8-18 carbon atoms and x, y and z are numbers ranging from 2 to 50, and quaternary ammonium salts having a formula of: 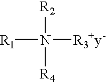
where R₁ and R₂ are an alkyl, alkyl-aryl-alkyl, ethoxyalkyl, hydroxyalkyl, or ethoxylated alkylphenol with a 4 to 22 carbon atom chain and the ethoxylated alkyls having 2 to 15 ethoxyl groups, and R₃ and R₄ are alkyl groups having a 1 to 15 carbon atom chain and y, for example, a halogen, sulphate, sulphite or phosphate.

Combinations of the aforesaid amines and quaternary ammonium salts are particularly advantageous, since such combinations facilitate a precise release rate for the nucleotides. Such surface active agents also include hyamine chloride, that is, diisobutyl cresoxy ethoxy ethyl dimethyl ammonium chloride.

Ethoxylated amines release nucleotides quantitatively from microbes, but the quaternary ammonium salts of ethoxylated amines penetrate the cell wall of microbes faster and quaternary salts are affected less by buffers, pH, and other agents possibly encountered in the sample than are ethoxylated amines. A 0.02-0.5% solution of a mixture of 1 to 19 parts of an ethoxylated amine, and 1 to 19 parts of a quaternary ammonium salt of an ethoxylated amine, both having a carbon atom chain length from 8 to 18, provide a complete and rapid release of nucleotides from bacteria, yeasts, fungi, and slime molds as well as from certain bluegreen, green, brown and red algae. Nucleotides are also released from somatic cells with these reagents, but due to the precipitation of some proteins by the reagents, the release can be incomplete. These reagents do not inhibit firefly luciferase in the degree that would interfere with the measurement. On the contrary, the presence of a low concentration of a quaternary ethoxylated amine (0.001-0.03%) enhances the turnover rate of the firefly luciferase and produces up to about twice as many photons per second during the first part of the reaction as is produced by the same concentration of ATP with the same luciferin-luciferase reagents in plain buffer solution.

The rate of release of ATP and the bioluminescent reaction are affected by the proportions of the ethoxylated amine and the quaternary salt of the ethoxylated amine. The release of ATP and other nucleotides from microbial cells is slow, but still quantitative, and the reaction rate of luciferase is the same as in buffer when ethoxylated amines are used for the release. By including a quaternary salt of an ethoxylated amine with the ethoxylated amine the rate of nucleotide release and the bioluminescent reaction can be increased from moderately slow to moderately fast depending on the proportion of the quaternary ethoxylated amine in the reagent.

The accompanying Figure illustrates the effect of the proportion of quaternary ethoxylated amine on the reaction kinetics and level of light emulsion as a function of time after adding ionic surface active agent in bacterial sample. Numbers refer to the percentage of ethoxylated amine and quarternary ethoxylated amine, respectively in a total concentration of 0.1% in the sample. Both ethoxylated amine and quarternary ethoxylated amine had a chain length of 12 carbons. “A” refers to the time of adding the surface active agent and “B” to the time of adding the firefly reagent. In the tests 100 microliters of 0.2% aqueous solution of the ethoxylated amine and a quaternary ethoxylated amine were pipetted to 100 microliters volume of bacterial suspension (E. coli) and the sample was mixed by shaking for fifteen seconds before being measured in a photometer where 100 microliters of luciferin-luciferase reagent was injected to the sample in a light-tight reaction chamber just prior to the measurement. The ethoxylated amine alone releases ATP from bacteria in 15 seconds and produces the same reaction kinetics as ATP in plain buffer, that is a continuous emission of light that decays 1 to 10% per minute depending on the proportions of the components in the luciferin-luciferase reagent.

The quaternary salt of an ethoxylated amine used alone causes too fast a reaction rate to allow easy and reproducible measurement of ATP. A mixture of the ethoxylated amine and a quaternary salt of an ethoxylated amine, having both a 12 carbon chain length, gives the possibility to select a desired rate of nucleotide release and reaction kinetics. As can be seen in the Figure, the level of light emission is increased about two times by the use quaternary salt over the ethoxylated amine alone as a releasing agent. More than one half of a quaternary salt of an ethoxylated amine in the reagent causes inactivation of the luciferase enzyme; thus the proportions have to be controlled.

While ethoxylated amines do release nucleotides reasonably rapidly from bacteria, they produce a slow release from yeasts and fungi which have an even stronger cell wall than bacteria. Therefore, it is beneficial to include a quaternary salt of an ethoxylated amine as part of the release reagent. One third of a quaternary ethoxylated amine and two thirds of ethoxylated amine give a complete release of nucleotides, such as ATP and FMN from most bacteria in a few seconds, and from Mycobacteria, yeasts and fungi in 30-60 seconds.

If the sample contains microbial cells only, or if the proportion of non-microbial cells is insignificant (for example, activated sludge, soil, sediments), the release of nucleotides from the sample is accomplished by simply adding, for example a mixture of an ethoxylated amine and a quaternary ethoxylated amine in an end-concentration of 0.05-0.5% by volume of the sample. The reagent and sample are mixed and the reagent is allowed to remain in contact with the cells for a sufficient time to complete the release of nucleotides. After this the sample should be measured within five minutes to avoid any breakdown of nucleotides by the enzymes left inside the cells. These ionic reagents do inactivate some enzymes, among which is photobacterial luciferase. Therefore, the sample should be diluted 5 to 100 times after treatment and before measurement if FMN or pyridine nucleotides are measured in the sample with the bacterial bioluminescent system. The nucleotide release reagent for microbial cells, consisting of ethoxylated amines, can be adjusted to acidic and alkaline pH; thus the reduced and oxidized pyridine nucleotides can selectively be extracted from microbial cells.

The release reagent for microbial nucleotides requires a direct contact with the cell wall, in order to give a quantitative release. Therefore, the cells have to be in suspension. If cells form clumps or flocculates in the sample, they should preferably be dispersed prior to application of the reagent. The dispersion or homogenizing must not stress the cells because stress would affect the level of nucleotides in the cells.

Table 1, below, shows that a linear relationship is obtained between the number of bacterial cells and the concentration of released ATP when a 3:7 mixture of an ethoxylated quaternary amine and ethoxylated amine were used to extract this purine nucleotide from E. coli suspension:

TABLE 1

| | Relative light units for 10 second |
|---|---|
| Number of bacteria per milliliter | integration |
| 150,000 | 1,480 |
| 500,000 | 4,800 |
| 1,000,000 | 9,580 |
| 2,000,000 | 18,900 |

The procedure was as follows: 100 microliters of 0.2% releasing reagent was added to 100 microliters of sample and the solution was mixed for 15 seconds. The sample was placed in the light-tight reaction chamber of the photon counter. Just prior to starting a 10-second integration, 100 microliters of firefly luciferin-luciferase reagent was added to the sample in a transparent cuvette in the reaction chamber. The bacteria were grown on a liquid nutrient medium and the number of cells was determined by standard colony counting. The different dilutions were made in physiological saline solution.

The extraction efficiency of the release reagent based on a quaternary ethoxylated amine and an ethoxylated amine mixture was also tested against the boiling tris-EDTA buffer and the perchloric acid extraction methods. The samples consisted of whole blood diluted with physiological saline 200 times, a suspension of E. coli bacteria in concentration of one million cells per milliliter, and a suspension of green algae, Chlorella sp.

Sample treatments were:
Ionic surface active nucleotide releasing reagent: To a 100 microliter aliquot of sample solution, 100 microliters of a 0.2% aqueous solution of a 3:7 part mixture of an ethoxylated quaternary amine and ethoxylated amine was pipetted and mixed for 15 seconds (for blood and bacteria) or 60 seconds (for algae).
Perchloric acid method: 0.1 ml 1 N perchloric acid was pipetted to 1 ml of sample and mixed for one minute. Sample was neutralized by adding 0.1 ml 1 N NaOH.
Boiling tris-EDTA method: Nine milliliters of tris (0.02 molar)-EDTA (0.002 molar) at pH 7.4 was heated to boiling and 1 ml of sample solution was pipetted on the boiling buffer. Samples were boiled for three minutes and cooled on ice.

Measurement of ATP by bioluminescence: 100 microliter aliquots of extracted sample solution were pipetted into transparent glass cuvettes and these were placed into the light-tight reaction chamber of a photon counter. Just prior to the measurement, 100 microliters of firefly luciferin-luciferase reagent was injected into the sample. The light emission was integrated for 10 seconds and the results read on the digital display as relative light units. The relative light units were converted to ATP by internal standardization whereby a known quantity of ATP standard was added into an aliquot of the extracted sample and measured as above. The standard was added in 10 microliters in order not to change the total volume of the sample significantly. By subtracting the reading of the sample from that of the sample+added ATP standard a conversion factor for relative light units to ATP was calculated. The results of the extraction efficiency test are given in Table 2, below:

TABLE 2

| | Ionic surface | | |
|---|---|---|---|
| | active nucleotide | Perchloric | Tris-EDTA |
| Sample | releasing agent | acid method | boiling buffer |
| Whole blood | 100% | 7% | 80% |
| E. Coli suspension | 100% | 3% | 95% |
| Chlorella sp., alga | 100% | — | 100% |

In all sample types the ionic surface active reagent gave the highest extraction efficiency. Boiling tris-EDTA buffer method extracted the same quantity of ATP from algae, but less from bacteria and blood. The low values obtained with perchloric acid method are due to co-precipitation of ATP with the perchlorate during the neutralization step. Perchloric ion is also a strong inhibitor of the firefly bioluminescent reaction.

### EXAMPLES

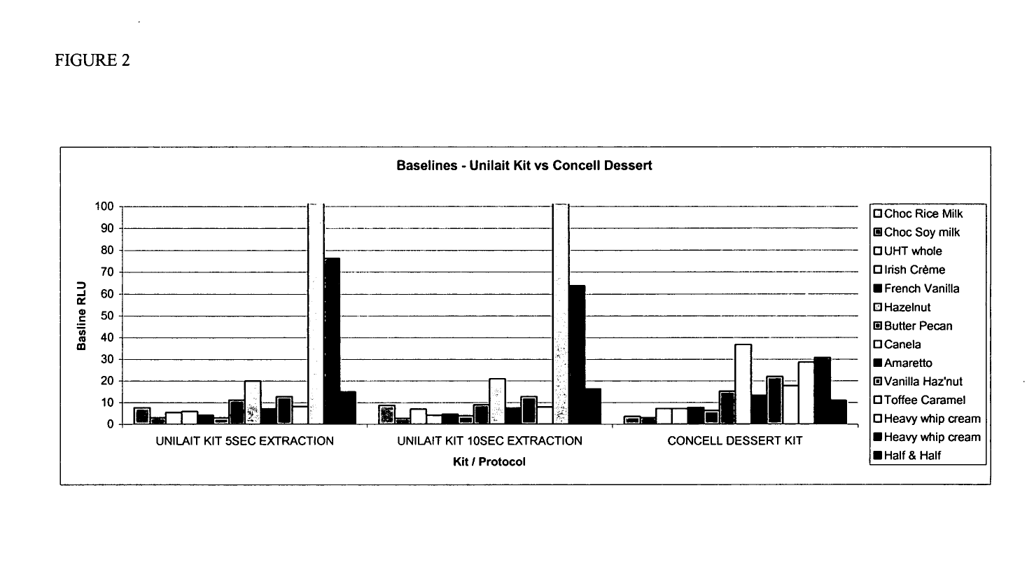

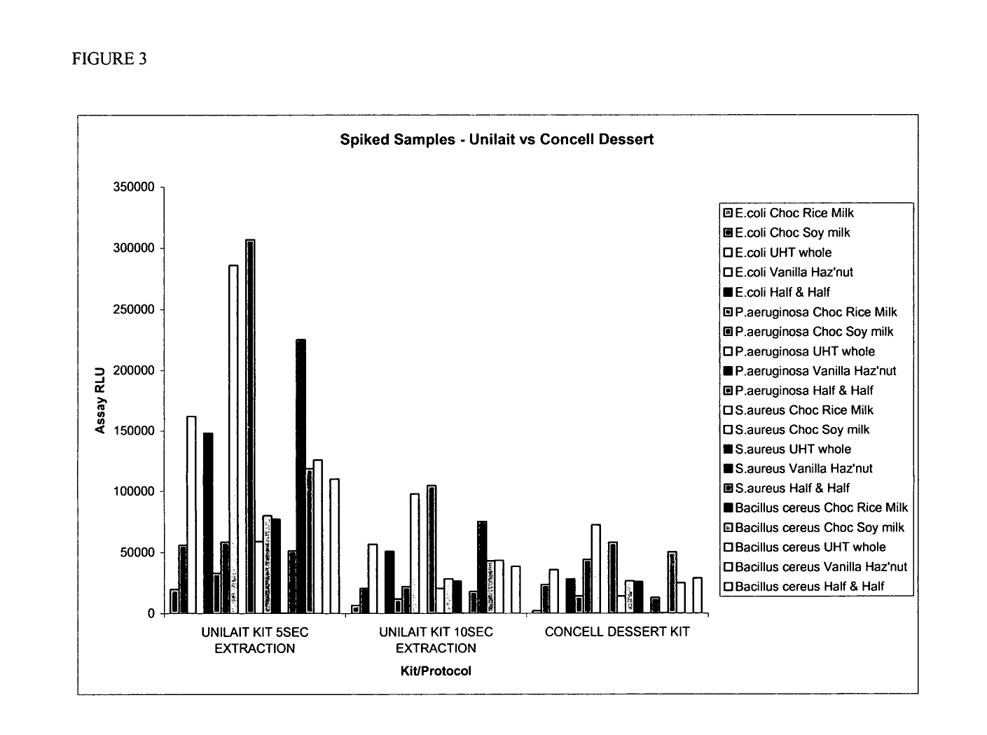

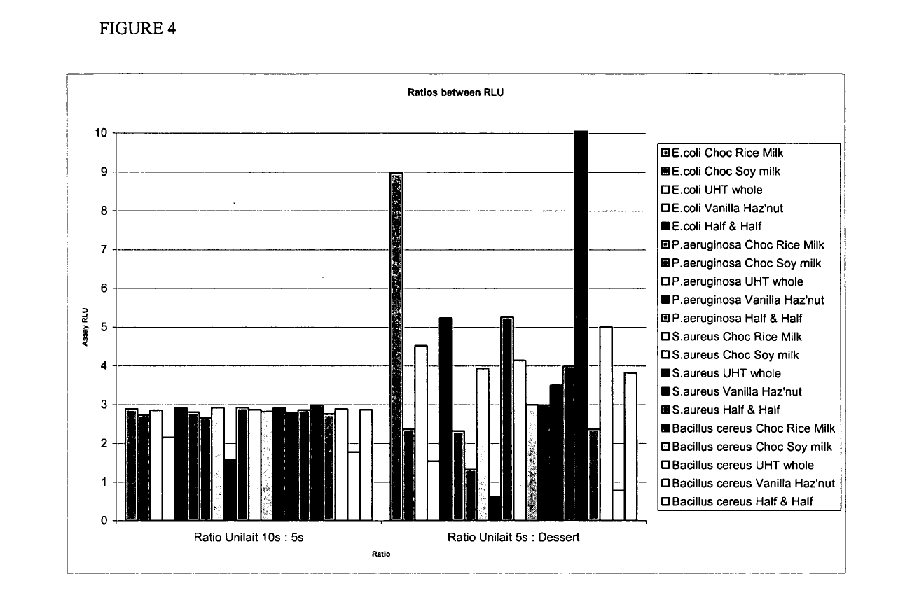

Appendix A shows comparative results of bacterially spiked samples between an older dairy products test, using less effective releasing agents (Dessert Kit available from Celsis, Inc., 400 West Erie, Suite 300, Chicago Ill. 60610-6910 USA) compared to a new test in accordance with the present invention, using a 0.8 percent aqueous solution of didodecyl dimethyl ammonium chloride as an extractant or releasing reagent. These spiked samples were then measured. As a test sample various milk based dairy products were spiked with a number of bacterial strains. 50 microliters of milk based dairy product was incubated with 25 microliters of an apyrase enzyme solution in order to reduce non-microbial ATP, followed by an injection of 50 microliters of extractant. The samples were then allowed to stand for 5 seconds and 100 microliters of Luciferase—Luciferin was injected into the sample. The light signal was then recorded and expressed as relative light units (RLU). FIG. 2 illustrates the baselines for a prior art kit verses a kit in accordance with the present invention. FIG. 3 illustrates the spiked samples for a prior art kit verses a kit in accordance with the present invention. FIG. 4 illustrates ratios between relative light units for a prior art kit verses a kit in accordance with the present invention.

Appendix B is similar to Appendix A and gives a comparison between a current, commercially available test (Biotrace kit available from Biotrace International Plc., The Science Park, Bridgend, Wales CF31 3NA United Kingdom) and a new test in accordance with the present invention, which uses halide containing quaternary ammonium compounds as releasing agents as disclosed in this application.

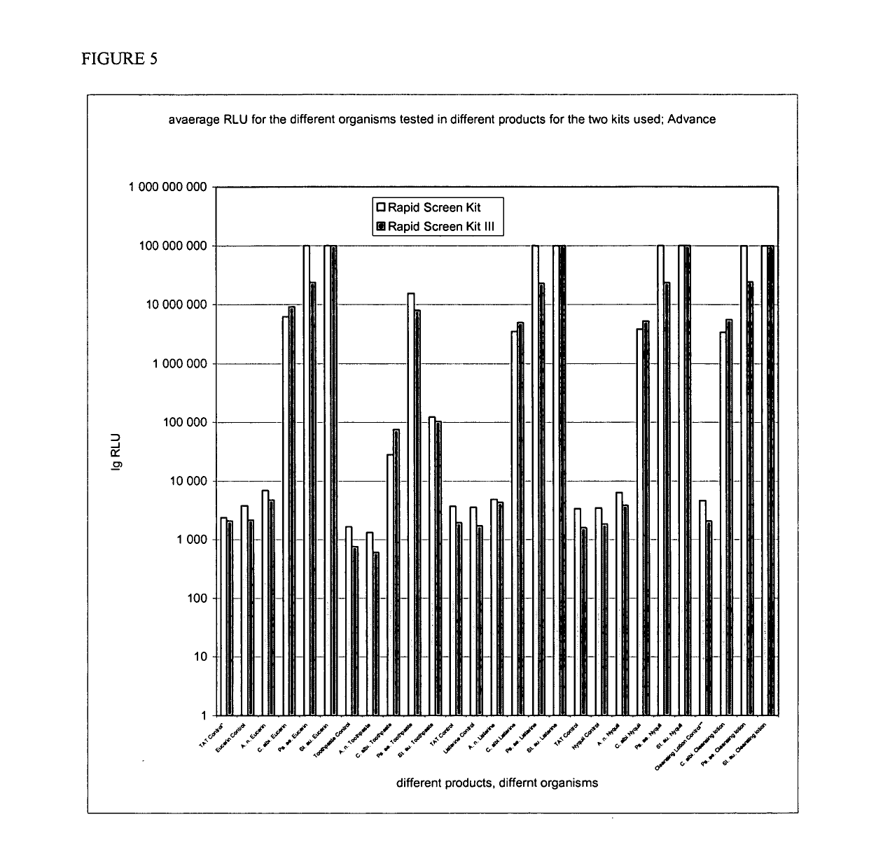

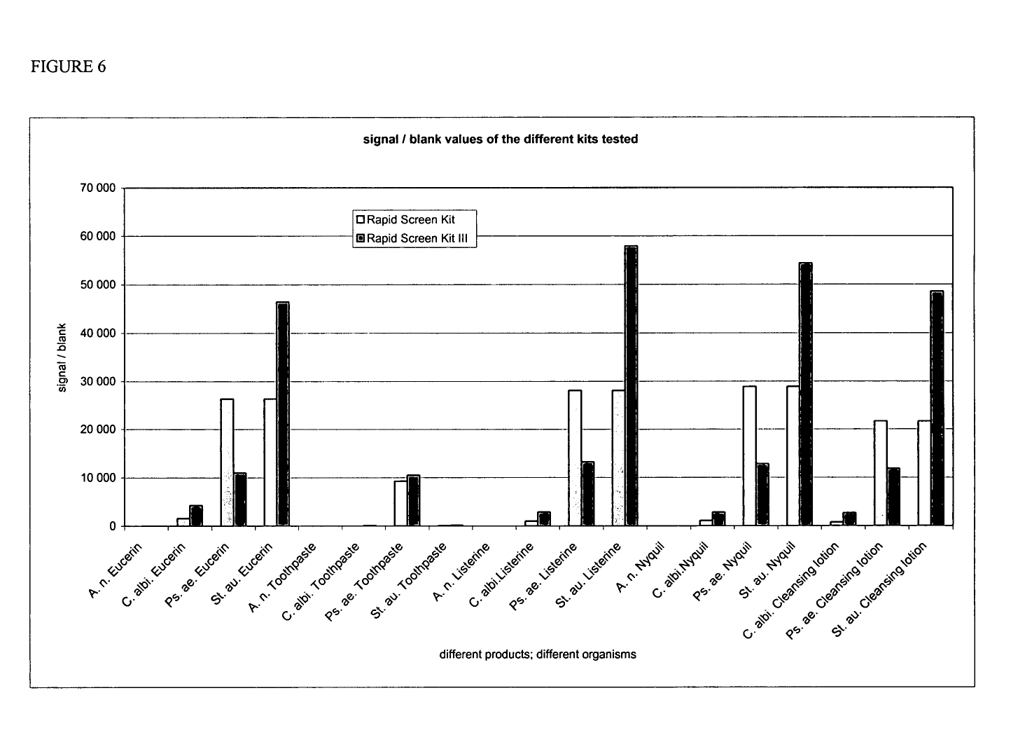

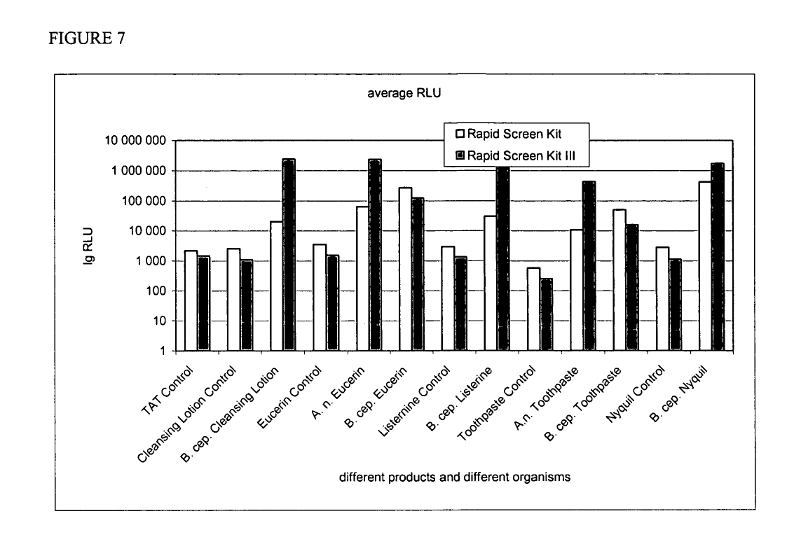

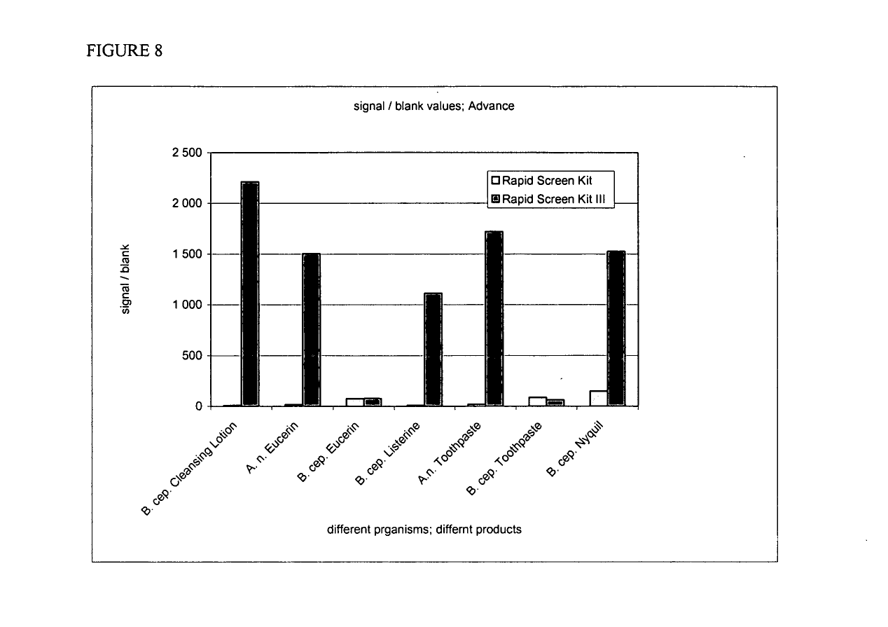

Appendix C also gives a comparison between an older test (Rapid Screen Kit available from Celsis, Inc.) and a new test in accordance with the present invention, which uses halide containing quaternary ammonium compounds as releasing agents. Specifically, this Appendix shows test results for non-dairy types of samples incubated over 16 hours in a nutrient solution in order to allow microbes in the samples to multiply. The results are expressed as ratio of the light signal divided by the background noise signal—this ratio is used regularly to show differences in detection sensitivity. FIG. 5 illustrates the average relative light units for the different organisms tested in different products for a prior art kit verses a kit in accordance with the present invention. FIG. 6 illustrates the signal/blank values for a prior art kit verses a kit in accordance with the present invention. FIG. 7 illustrates the average relative light units for the different organisms tested in different products for a prior art kit verses a kit in accordance with the present invention. FIG. 8 illustrates the signal/blank values for a prior art kit verses a kit in accordance with the present invention. This incubation technique is common for products such as shampoos and tooth pastes, which have lower numbers of microbes than other products. Incubation of such samples is a multiplying technique which allows a low number of microbes to be increased and thereby made more easily detectable. The new kit allows for much better and faster detection of bacterial strains which grow slowly such as Burkholderia and Candida.

Appendix D is similar to Appendix C except that the results are expressed as relative light units instead of signal to blank ratio as in Appendix C.

Appendix E describes the use of didodecyl dimethyl ammonium bromide as a selective releasing reagent for Gram negative bacteria in presence of yeast cells.

While the invention has been described with specific embodiments, other alternatives, modifications and variations will be apparent to those skilled in the art. Accordingly, it will be intended to include all such alternatives, modifications and variations set forth within the spirit and scope of the appended claims.

1. A method for releasing cellular contents comprising using a halide containing quaternary ammonium compounds.

2. The method of claim 1 further including releasing molecules of interest from living cells.

3. The method of claim 1 further including releasing nucleotides from living cells.

4. The method of claim 1 further comprising applying ionic surface active agents containing a fatty group with a chain length of from 8 to 18 carbon atoms.

5. The method of claim 4 further comprising applying ionic surface active agents containing a fatty group with a chain length of 12 carbon atoms.

6. A method for releasing cellular contents comprising modifying the alkyl chain length of ionic surface active agents to enable selective release from different classes of living cells and applying the ionic surface active agents having different chain lengths to effect to change the permeability of the cell wall and membranes.

7. The method for releasing cellular contents of claim 6 further comprising applying anionic surface active agents to the exclusion of non-ionic surface active agents.

8. The method for releasing cellular contents of claim 6 further comprising applying cationic surface active agents to the exclusion of non-ionic surface active agents.

9. The method for releasing cellular contents of claim 6 further comprising applying ionic surface active agents containing quaternary ammonium salts.

10. The method for releasing cellular contents of claim 6 further comprising applying ionic surface active agents containing a fatty group with a chain length of from 8 to 18 carbon atoms.

11. The method for releasing cellular contents of claim 10 further comprising applying ionic surface active agents containing a fatty group with a chain length of 12 carbon atoms.

12. The method for releasing cellular contents of claim 6 further comprising applying ionic surfactants selected from the group comprising: ethoxylated amines; ethoxylated diamines; polyethylene glycol esters of fatty acids; ethoxylated amides having a chemical structure of: 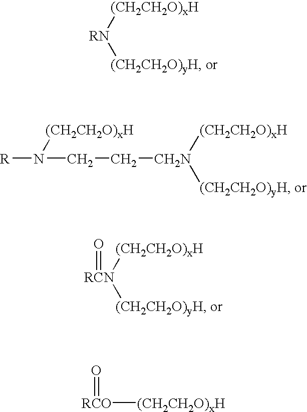
where R is a fatty alkyl group having 8-18 carbon atoms and x, y and z are numbers ranging from 2 to 50; quaternary ammonium salts having a formula of: 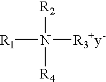
where R₁ and R₂ are an alkyl, alkyl-aryl-alkyl, ethoxyalkyl, hydroxyalkyl, or ethoxylated alkylphenol with a 4 to 22 carbon atom chain and the ethoxylated alkyls having 2 to 15 ethoxyl groups, and R₃ and R₄ are alkyl groups having a 1 to 15 carbon atom chain and y is selected from the group comprising a halogen, sulphate, sulphite, phosphate and combinations thereof; and combinations thereof.

13. The method for releasing cellular contents of claim 6 further comprising applying hyamine chloride.

14. The method for releasing cellular contents of claim 13 further comprising applying diisobutyl cresoxy ethoxy ethyl dimethyl ammonium chloride.

15. The method for releasing cellular contents of claim 6 further comprising applying a mixture of an ethoxylated amine and a quaternary salt of an ethoxylated amine.

16. The method for releasing cellular contents of claim 15 further comprising applying a 0.02-0.5% solution of a mixture of 1 to 19 parts of an ethoxylated amine, and 1 to 19 parts of a quaternary ammonium salt of an ethoxylated amine, both having a carbon atom chain length from 8 to 18.

17. The method for releasing cellular contents of claim 15 further comprising applying a mixture of ethoxylated amine and a quaternary salt of an ethoxylated amine, having both a 12 carbon chain length.

18. The method for releasing cellular contents of claim 15 further comprising applying a mixture of one third of a quaternary ethoxylated amine and two thirds of ethoxylated amine.

19. The method for releasing cellular contents of claim 15 further comprising applying a mixture of a mixture of an ethoxylated amine and a quaternary ethoxylated amine in an end-concentration of 0.05-0.5% by volume of the microbe sample.

20. A substance for effecting a selective release of cellular contents comprising a dialkyl ammonium halide.

21. The substance for effecting a selective release of cellular contents of claim 20 further wherein the dialkyl ammonium halide comprises an alkyl chain lengths of approximately 8 to 10 carbon atoms.

22. The substance for effecting a selective release of cellular contents of claim 20 further wherein the dialkyl ammonium halide comprises an alkyl chain lengths of approximately 10 carbon atoms or greater.

23. A substance for effecting a selective release of cellular contents comprising a dimethyl ammonium halide.

24. The substance for effecting a selective release of cellular contents of claim 23 further wherein the dimethyl ammonium halide comprises an alkyl chain lengths of approximately 8 to 10 carbon atoms.

25. The substance for effecting a selective release of cellular contents of claim 23 further wherein the dimethyl ammonium halide comprises an alkyl chain lengths of approximately 10 carbon atoms or greater.
